(19) United States
(12) Patent Application Publication
(10) Pub. No.: US 2002/0029037 A1
(43) Pub. Date: Mar. 7, 2002

(54) Method and apparatus for percutaneous trans-endocardial reperfusion

(76) Inventor: Young D. Kim, McLean, VA (US)

Correspondence Address: George E. Quillin, FOLEY & LARDNER, Washington Harbour, 3000 K Street, N.W., Suite 500, Washington, DC, 20007-5109, US

(21) Appl. No.: 09/946,928

(22) Filed: Sep. 6, 2001

Related U.S. Application Data
(60) Provisional application No. 60/230,440, filed on Sep. 6, 2000.

(51) Int. Cl. A61B 18/18

(52) U.S. Cl. 606/41

(57) ABSTRACT

A percutaneous trans-endocardial reperfusion catheter, and a method of using the same, is provided for the treatment of acute myocardial ischemia. The catheter includes a compressive, ferromagnetic, and electrically conductive tip which is used with an external magnetic device to anchor the catheter tip in a desired position. Needles are movably mounted within the catheter to extend through exit ports formed therein near the catheter's distal end. When extended, the needles protrude beyond the ferromagnetic tip. With the ferromagnetic tip anchored in the myocardium, the needles may be extended to penetrate the tissue to form channels. One or more of the needles may have a hollow channel and holes formed therein for the delivery of drugs, blood, or other fluids directly into the tissue in which the needle is imbedded. An electrode positioned at the catheter tip may be used to detect electrical signals and to provide electrical therapy.

### BACKGROUND OF THE INVENTION

This application claims priority to United States Provisional Patent Application Serial No. 60/230,440 filed Sep. 6, 2000.

1. Field of the Invention

The present invention pertains generally to medical methods and devices. More particularly, this invention relates to medical catheters, and to methods and devices for reestablishing blood flow to an ischemic myocardium, including methods and devices for forming holes or channels into the myocardium.

2. Description of the Related Art

Acute myocardial ischemia/infarction (hereinafter “acute myocardial ischemia”) is an event resulting from a sudden blockage of an epicardial coronary artery, i.e., of a conduit artery carrying blood to the heart muscle (hereinafter “myocardium”). Typically, the blockage of such epicardial arteries can be attributed to a sudden rupture of a plaque and/or intravascular thrombosis, i.e., clotting within a blood vessel. If reperfusion (i.e., the restoration of blood flow to the ischemic portion of the myocardium) is not achieved in a timely manner, at least a portion of the myocardium may be permanently damaged, thereby permanently and adversely affecting a patient's cardiac performance and, in some cases, resulting in a patient's death.

For more than a decade, intravenous thrombolytic therapy has been the standard reperfusion therapy for patients with acute myocardial ischemia. Such therapy may involve, for example, the injection of an anti-clotting agent in an attempt to break up or dissolve a thrombus blocking an epicardial coronary artery. Such a therapy is typically applied within 6 to 12 hours (hereinafter “critical time”) of symptom onset. Thrombolytic therapy has several major limitations. The failure rate, even with the most potent thrombolytic therapy, is about 50 percent. Thrombolytic therapy results in a relatively high rate (about 1 in 150 to 200 treatments) of intracranial hemorrhage, which is usually fatal. Moreover, the eligibility rate for thrombolytic therapy, based on current practice criteria, is only approximately 16 to 33 percent.

To overcome the above inherent limitations of thrombolytic therapy, alternative reperfusion therapies, such as percutaneous transluminal coronary angioplasty and emergency coronary arterial bypass graft surgery, have been adapted to replace and/or supplement thrombolytic therapy for acute myocardial ischemia. However, both percutaneous transluminal coronary angioplasty and coronary arterial bypass graft surgery are relatively complicated and/or require major surgery. In addition, it is estimated that less than 20 percent of primary care hospitals in the United States are capable of instituting percutaneous transluminal coronary angioplasty and/or coronary arterial bypass graft surgery for patients suffering from acute myocardial ischemia. Thus, these patients must be transferred to secondary or tertiary hospitals to receive such interventional reperfusion therapy. In addition, percutaneous transluminal coronary angioplasty and coronary arterial bypass graft surgery are themselves technically difficult and time-consuming procedures, as compared with thrombolytic therapy. In most cases, as a result of patient transfer or the time required to perform the procedure itself, therefore, myocardial reperfusion by percutaneous transluminal coronary angioplasty and/or coronary arterial bypass graft surgery may be delayed beyond the critical time and, therefore, regardless of the high success rate of reperfusion, the myocardium may suffer permanent damage.

Clinically and experimentally, it is well documented that the time lapsed from the onset of acute myocardial ischemia to reperfusion is a major determining factor for the efficacy of reperfusion therapy independent of the mode of reperfusion. Generally, reperfusion should be established within the critical time to have benefits. Unfortunately, in most clinical situations, the current mechanical reperfusion therapies, such as percutaneous transluminal coronary angioplasty and/or coronary arterial bypass graft surgery, are not readily available within the critical time resulting in a delay and/or underutilization of reperfusion therapy. As such, the mortality rate for acute myocardial ischemia remains high.

It has been noted that reperfusion of the ischemic myocardium in end stage coronary arterial disease may be established directly through the formation of intra-myocardial holes or channels, rather than through procedures involving the native coronary arteries, such as percutaneous transluminal coronary angioplasty and coronary arterial bypass graft surgery. Examples of such reperfusion therapy include percutaneous transendocardial reperfusion and trans-myocardial revascularization.

In percutaneous trans-endocardial reperfusion, intra-myocardial channels are created trans-endocardially (i.e., from the inside of the heart). In contrast, a trans-epicardial (i.e., from outside the heart) transmural approach is used in trans-myocardial revascularization. U.S. Pat. Nos. 5,725,521 and 5,878,751 disclose the effectiveness of mechanical transmural acupuncture in the treatment of acute myocardial ischemia (such as using needle acupuncture to create channels for the delivery of oxygenated blood into myocardial tissue). However, percutaneous trans-endocardial reperfusion and transmyocardial revascularization are both typically performed as highly technical, laser-based procedures.

Several patents discuss the use of lasers for forming multiple channels in the myocardium using a catheter which has been placed in a desired position in the heart. For example, U.S. Pat. No. 5,769,843 describes steering a laser beam directed out of a hole formed in the distal end of a catheter to cut multiple channels in the myocardium without moving the catheter. U.S. Pat. No. 5,725,521 describes a catheter having a distal end with a pointed tip for piercing tissue to position the end of the catheter, and a plurality of laser delivery means (optical fibers/wave guides) extending distally and radially from near the end of the catheter.

Currently, laser based percutaneous trans-endocardial reperfusion and transmyocardial revascularization are typically used to treat patients with chronic, end stage inoperable coronary artery disease, rather than patients suffering from acute myocardial ischemia. Neither laser based trans-myocardial revascularization nor percutaneous trans-endocardial reperfusion is suitable for use as a tool for reperfusion therapy for acute myocardial ischemia patients; this is mainly because of their cumbersome natures and the inherent requirements of the high technology involved. It is likely that such laser-based revascularization techniques will be available only at highly specialized cardiac centers. Thus, the potential benefits of such therapies for the treatment of acute myocardial ischemia will not be realized unless methods and devices for employing such therapies can be simplified for use in almost all primary care facilities.

For the treatment of acute myocardial ischemia, percutaneous trans-endocardial reperfusion has several advantages as compared to trans-myocardial revascularization. As a percutaneous trans-endocardial reperfusion catheter can be introduced percutaneously into the left ventricular cavity via a femoral artery, percutaneous trans-endocardial reperfusion does not require general anesthesia or a thoracotomy. This percutaneous technique is relatively simple, less time consuming, and eliminates complications such as bleeding and hemodynamic instability. Additionally, percutaneous trans-endocardial reperfusion may be applicable to most of the regions of the myocardium, while trans-myocardial revascularization may be limited to epicardially accessible areas, such as anterior or lateral walls of the left ventricle.

Furthermore, both functionally and anatomically, it is anticipated that trans-endocardial channels created by percutaneous trans-endocardial reperfusion will relieve acute myocardial ischemia. Anatomically, it has been noted that, in acute myocardial ischemia, the pathological lesion is usually segmental, does not extend into small capillary arteries, and usually does not recruit collateral vessels because of the nature of its acute onset. Of particular note is that the micro-circulatory system (small distributing arteries and capillary vessels) of the blocked conduit artery is patent and functional if circulation is restored in time. This may be contrasted with the micro-circulatory system of end-stage coronary artery disease patients, which are characterized by the involvement of pathological processes well into small arteries.

Functionally, it is noted that local factors, as well as the systemic perfusion pressure, regulate blood flow to an organ system. The organ perfusion pressure is determined by systemic arterial blood pressure and venous pressure (in general, organ tissue pressure is equal to venous pressure). Locally, depending on the specific function of the organ, vascular tone will be regulated by metabolic needs (auto regulation).

Myocardial circulation is unique in that blood flow is intimately influenced by intra-myocardial tissue pressure, which is much higher than systemic venous pressure. Therefore, intra-myocardial tissue pressure plays a major role in determining myocardial profusion pressure, particularly when local auto-regulatory function is abolished such as in ischemic myocardium. Intra-myocardial tissue pressure is closely related to regional myocardial contractile function, and constitutes the intraventricular pressure, which is the driving force for cardiac pumping. Intra-myocardial tissue pressure is not uniform throughout the ventricular wall, even in a normal heart; this indicates ventricular contractile function is not uniformly distributed.

Normally, systolic intra-myocardial tissue pressure is equal to or higher than systolic aortic pressure, while the diastolic intra-myocardial tissue pressure is about the same as left ventricular diastolic pressure, but substantially lower than aortic diastolic pressure. This fact offers a simple explanation for why coronary blood flow occurs mainly during the diastole. When a specific region of the myocardium becomes ischemic, the myocardium ceases to contract, and systolic intra-myocardial tissue pressure of that region of the ventricle decreases to near diastolic pressure thereby creating a significant perfusion pressure during systole as well as during diastole. For this reason, it is very likely that blood flow will be re-established if any channel is created between a ventricular cavity and the ischemic myocardium. Blood flow will be further enhanced if any channel is created between the systemic artery and the ischemic myocardium.

Although the long-term patency rate of intra-myocardial channels created by percutaneous trans-endocardial reperfusion (or trans-myocardial revascularization) is universally poor, short-term improvements of symptoms and/or blood supply to the myocardium are obtainable. Thus, although percutaneous trans-endocardial reperfusion may not provide a long-term solution to acute myocardial ischemia, percutaneous trans-endocardial reperfusion may be used as a temporary “bridge” reperfusion therapy for acute myocardial ischemia. This temporary reperfusion therapy may be used primarily to provide a safe alternative treatment until a definitive revascularization therapy, such as percutaneous transluminal coronary angioplasty, coronary stenting, or coronary arterial bypass graft surgery is available.

Use of percutaneous trans-endocardial reperfusion for the treatment of acute myocardial ischemia requires the location of the area of the myocardium to be treated. U.S. Pat. No. 5,769,843 discloses using electrodes on laser catheters to detect conduction tissues, which allow location of the tissue to be treated. U.S. Pat. Nos. 5,431,640, 5,769,843, 5,895,404, and 5,902,238 disclose the use of magnets mounted in the distal end of a catheter for various purposes, including detecting the position and orientation of the distal end of the catheter and directing the catheter to a desired position. In particular, fixing the distal end of a catheter in a desired location by using a magnet mounted on the distal end of the catheter and an external magnet (employed to provide a magnetic force to position the end of the catheter) is discussed in U.S. Pat. Nos. 4,809,713 and 5,904,147.

In U.S. Pat. No. 4,809,713, conduction for pacing or other excitation of the heart is provided by an electrode which is positioned on the end of a catheter distally of a magnet used for fixation, or which is brought out through a channel formed in the magnet. In addition to magnets, U.S. Pat. Nos. 3,754,555, 3,976,082, 5,507,802, and 5,693,081 disclose using prongs, fibers, bristles, etc. (which may extend out of apertures formed near the distal end of a catheter into adjacent tissue) to secure the end of a catheter in position. Moreover, U.S. Pat. Nos. 5,725,521 and 5,904,147 disclose the use of catheters for the infusion of therapeutic drugs.

What is desired is an apparatus and method for performing percutaneous trans-endocardial reperfusion in a timely and relatively easy manner, such that percutaneous trans-endocardial reperfusion may be used in the treatment of acute myocardial ischemia in most primary care hospitals. Such a percutaneous trans-endocardial reperfusion apparatus should be both inexpensive and easy to use, while providing a physician with the necessary features for determining the proper location for therapy, for directing and positioning the apparatus in the proper location for applying such therapy, and, preferably, for providing a variety of electrical and/or chemical therapies, in addition to reperfusion, when the apparatus is in position.

### SUMMARY OF THE INVENTION

The present invention provides a percutaneous trans-endocardial reperfusion method and apparatus. In particular, the invention address a percutaneous trans-endocardial reperfusion catheter which is relatively inexpensive and easy to use and which may be used to provide a variety of therapies. A percutaneous trans-endocardial reperfusion catheter in accordance with the present invention is particularly applicable for use in treating patients suffering from acute myocardial ischemia. A percutaneous trans-endocardial reperfusion catheter in accordance with the present invention is designed to be introduced into a patient's heart percutaneously, and to be used to create channels through the endocardium to reestablish blood flow to an ischemic myocardium. The aim of such reperfusion therapy in accordance with the present invention is to prevent permanent myocardial damage until definitive reperfusion therapy is available.

As the technique of employing the method and apparatus of the present invention for the treatment of acute myocardial ischemia is relatively easy and safe, the present invention may be employed for the treatment of acute myocardial ischemia at most primary care hospitals. A percutaneous trans-endocardial reperfusion catheter in accordance with the present invention may incorporate features such as: (a) facilitating the location of ischemic regions of the myocardium requiring treatment; (b) positioning the catheter in a desired position at which treatment may be provided; (c) mechanically creating a plurality of channels through the endocardium and into the myocardium to reestablish blood flow; (d) electrically sensing, pacing, and/or defibrillating the heart, as required; and (e) directly delivering drugs or blood (i.e., augmenting reperfusion) into targeted areas of the myocardium. Some or all of these features of the present invention may be incorporated into a catheter in accordance with the present invention.

A catheter in accordance with the present invention includes a catheter body having a distal end and a proximal end. A ferromagnetic material is preferably mounted at the distal end of the catheter body. The ferromagnetic material is preferably compressive and electrically conductive and may be formed, for example, of a sponge material. In operation, the ferromagnetic tip of the catheter is placed in direct contact with the endocardium or other tissue while an external magnetic system is activated. With sufficient magnetic force applied, the tip of the catheter will be compressed firmly against the endocardium to anchor the tip of the catheter in place. The ferromagnetic material may have several slits formed therein, such that the ferromagnetic tip is splayed open when the tip is magnetically compressed against the endocardium or other tissue.

An electrode, which may be a platinum disk, may be mounted in the distal end of the catheter adjacent to the ferromagnetic tip. A wire conductor, which is preferably paramagnetic or diamagnetic, may be connected to and extend from the electrode through the catheter body to the proximal end of the catheter body. The electrode may be electronically coupled via the wire conductor to a device for detecting cardiac electrical signals such as an EKG or monophasic action potential detector. The electrode may similarly be electronically coupled via the wire conductor to devices for applying pacing and/or defibrillating electrical energy to the heart.

The electrode may be positioned within the catheter body adjacent and proximal to the ferromagnetic tip, or it may be positioned outside of the catheter on the distal end of the ferromagnetic tip. In the former case, signals may be detected by the electrode through the electrically conductive ferromagnetic tip. In addition, pacing and/or defibrillating energy may applied to the heart via the electrode through the electrically conductive ferromagnetic tip. In the latter case, a proximal side of the electrode, which is in contact with the ferromagnetic tip, may be shaped so as to form a wedge which facilitates splaying open the ferromagnetic material when the catheter tip is compressed against the endocardium or other tissue thereby driving the electrode proximally into the ferromagnetic material.

At least one exit port (and preferably a plurality of exit ports) is preferably formed in the catheter body near the distal end of the catheter body. Moreover, the at least one exist port is preferably slightly proximal of the ferromagnetic tip. One or more needles are moveably mounted in the catheter body. The needles are mounted in the catheter body so as to be aligned with the exit ports such that when the needles are moved forward the needles will extend through the exit ports forward of the distal end of the catheter. The needles are preferably formed of a relatively rigid material, such as plastic. In addition, the needles are sufficiently long such that, when the tip of the catheter is positioned against the endocardium and the needles are extended from the exit ports, the needles will extend into the myocardium to form channels therein.

Although the needles may be manually moved backward and forward, a reciprocator is preferably provided for moving the needles backward and forward, i.e., into and out of the catheter, such that the needles may be moved backward and forward into and out of the myocardium to form simultaneously a plurality of channels in the myocardium. Moreover, the reciprocator is preferably an oscillator. The needles may be mounted onto the distal side of a mobile disk which is mounted within the catheter; the disk may provide a sliding seal. A piston may be mounted at the proximal end of the catheter. The interior of the catheter (i.e. the “lumen”), between the mobile disk and the piston, may be filled with a fluid, such as heparinized normal saline. The forward movement of the piston generates positive pressure inside the catheter to move the mobile disk forward thereby advancing the needles. Correspondingly, the backward movement creates a negative pressure which retracts the mobile disk thereby pulling the needles into the catheter. Thus, the needles may be extended from and retracted into the catheter hydraulically.

One or more of the needles, which are extendable from the distal end of the catheter into the myocardium or other tissue, may have a channel formed therein. The needle channel may be in fluid communication with one or more holes formed in a side of the needle near the distal end of the needle. The channel formed in the needle may be connected, at the proximal end thereof, to the distal end of a tubule which extends from the needle through the catheter toward the proximal end of the catheter body. The tubule preferably terminates, at a proximal end thereof, in a tubule port, which extends out of the catheter body. The tubule port may be connected to a drug delivery system. Drugs, blood, or other fluid may be delivered through the tubule port, through the tubule, through the needle channel, and through the holes formed in the needle directly into the myocardium, or other tissue (when the needle is advanced from the catheter into such tissue). By connecting the tubule port to an infusion pump, an arterial line, or the ascending aorta, continuous blood flow directly into an ischemic myocardium may be provided through the tubule, the channel, and the holes in the needle.

A percutaneous trans-endocardial reperfusion catheter in accordance with the present invention is especially adapted for use in the treatment of acute myocardial ischemia. A standard percutaneous technique may be used to introduce the catheter into the left ventricle of a patient. The identification of a region of the left ventricle suffering from ischemia may be detected initially by a standard twelve lead EKG. The ferromagnetic tip of the percutaneous trans-endocardial reperfusion catheter may be guided toward the target area with the assistance of an external magnetic device. Thus, the external magnetic device, located on the surface of the patient's chest, may be used both to direct the catheter tip and to force the ferromagnetic tip of the catheter firmly against the targeted portion of the endocardium (to anchor the catheter tip in position).

An endocardial EKG may be obtained via an EKG system coupled to the electrode positioned at the distal end of the anchored catheter. The endocardial EKG signal may be used to confirm the ischemia of the specific region. The oscillator, or other mechanism, is then operated to extend the needles outward from the exit ports distally of the catheter tip so as to penetrate the myocardium to create channels therein. The tip of the catheter may then be relocated to another position on the endocardium, and additional channels formed therein. Additional channels covering sufficient endocardial areas affected by the ischemia may thus be created until ischemic symptoms and signs are relieved. While the piercing needles are engaged in the ischemic myocardium, drugs, blood, and/or other fluid may be injected into the myocardium via a tubule in the catheter which is in fluid communication with a channel and holes formed in one or more of the needles. Similarly, the tubule may be connected to an intra-arterial cannula to provide continuous blood reperfusion through the channel and the one or more holes formed in the needle.

A structural understanding of the aforementioned apparatus and method for percutaneous trans-endocardial reperfusion will be easier to appreciate when considering the detailed description in light of the figures hereafter described.

### BRIEF DESCRIPTION OF THE DRAWINGS

The accompanying figures, which are incorporated in and constitute a part of the specification, illustrate presently preferred embodiments of the invention. Together with the above general description and the following detailed description, the figures serve to explain the principles of the invention.

FIG. 1 is a cross-section of the distal end of an exemplary percutaneous trans-endocardial reperfusion catheter in accordance with the present invention;

FIG. 2 is a cross-section of the distal end of an exemplary percutaneous trans-endocardial reperfusion catheter of FIG. 1 as positioned for use against a endocardium and including a plurality of needles in an extended position such that the needles penetrate the myocardium to form channels therein;

FIG. 3 is a schematic illustration of the proximal end of the catheter of FIG. 1, showing various devices for detecting and providing electronic signals which may be connected thereto;

FIG. 4 is a schematic illustration of the proximal end of the catheter of FIG. 1, showing a hydraulic mechanism mounted thereto for extending and retracting needles mounted in the distal end of the catheter;

FIG. 5 is a cross-section of the distal end of a percutaneous transendocardial reperfusion catheter in accordance with a second embodiment of the present invention showing channels formed in movable needles extending from the distal end of the catheter into tissue, a tubule for carrying fluid to such channels, and holes formed in the needles which are in fluid communication with the channels formed therein for delivering such fluid into the tissue;

FIG. 6 is a schematic illustration of a portion of the catheter of FIG. 5, showing a tubule port formed in the catheter for providing fluid into the tubule for delivery to needles having channels formed therein positioned at the distal end of the catheter;

FIG. 7 is a schematic illustration of an alternative embodiment of a catheter in accordance with the present invention;

FIGS. 8-10 are cross-sections of the distal end of an alternative embodiment of a percutaneous trans-endocardial reperfusion catheter in accordance with the present invention; and

FIG. 11 is a schematic illustration of a portion of a human body and heart illustrating an exemplary use of a percutaneous trans-endocardial reperfusion catheter in accordance with the present invention for the treatment of acute myocardial ischemia.

### DETAILED DESCRIPTION OF THE INVENTION

The present invention provides a relatively inexpensive and easy to use percutaneous catheter, and method of using the same. A percutaneous catheter in accordance with the present invention may be especially adapted for use in percutaneous trans-endocardial reperfusion. In particular, as a catheter in accordance with the present invention is relatively easy to use, it may be used at most primary care hospitals in percutaneous trans-endocardial reperfusion applications for the treatment of acute myocardial ischemia. An exemplary catheter in accordance with the present invention will, therefore, be described in detail below with reference to this particular application. However, it should be understood that a catheter in accordance with the present invention includes many features which make the catheter useful for many applications other than percutaneous trans-endocardial reperfusion both in general and for the treatment of acute myocardial ischemia in particular. Thus, it should be understood that the present invention is not limited to such particular applications. Other exemplary applications of the present invention, such as defibrillation, pacing catheter, drug delivery, etc., will also be later discussed in detail.

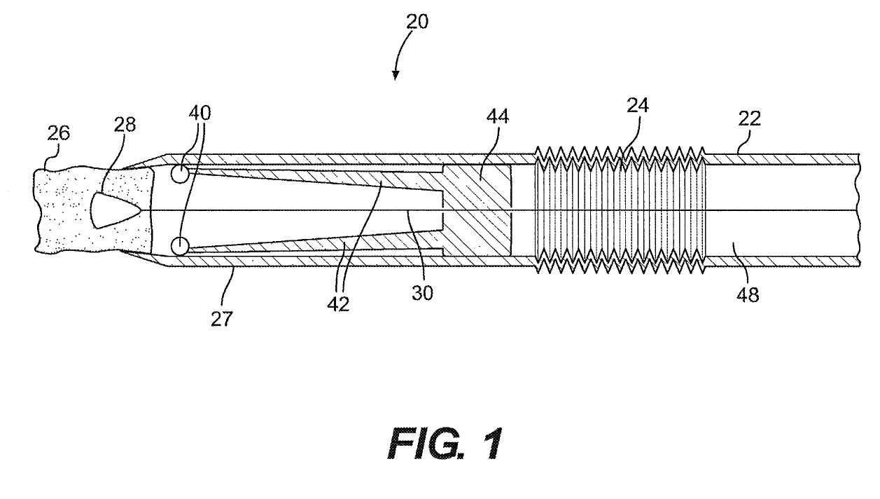

The distal end of an exemplary percutaneous trans-endocardial reperfusion catheter 20 in accordance with the present invention is illustrated in, and will be described in detail with reference to, FIG. 1. The catheter 20 includes a catheter body 22, which defines an interior and an exterior of the catheter 20. The catheter body 22 may be made of a conventional material such as polyethylene. The material forming the catheter body 22 may include a section 24 thereof, formed near the distal end 27 of the catheter 22, which is highly flexible, to allow the distal end 27 of the catheter 20 to bend easily. This “gooseneck” portion of the catheter body 22 may be implemented in a conventional manner. This highly flexible portion 24 of the catheter body 22 allows the distal end 27 of the catheter 20 to be bent at relatively sharp angles with respect to the rest of the catheter body 22. This allows for better positioning of the distal end 27 of the catheter 20.

As will be later discussed in more detail, a catheter 20 in accordance with the present invention may be introduced into the left ventricle 307 of a patient 300 through a femoral or brachial artery percutaneously. Thus, for example, the length of the catheter body 22 should be sufficient to reach a ventricle from, for example, a femoral artery. For a normal sized adult, a length of 120 centimeters for the catheter body 22 is sufficient. The outer diameter size of the catheter body 22 may preferably be about 7 or 8 French (i.e., about 2.3 millimeters which is about the same outer diameter as a regular adult sized Swan-Ganz catheter). Such a catheter size will require an 8 or 9 French standard percutaneous introducer system for introducing the catheter 20 into a blood vessel such as a femoral or brachial artery.

A ferromagnetic material is preferably positioned at the extreme distal end of the catheter 20, to form a tip 26 thereof. The tip 26 is preferably made of a compressive and electrically conductive, as well as ferromagnetic, material. By compressive, it is meant that the tip 26 is preferably soft and conformable. Moreover, the tip 26 may be penetrated by at least one sharp needle 42 extending therethrough. A preferred material for forming the tip 26 is a sponge material made of synthetic resins. An exemplary material for forming the ferromagnetic tip 26 is a polyurethane foam (Sunrise Medical, Bladwin Miss.) containing fine iron fibers (an iron sponge).

As will be discussed in more detail below, the ferromagnetic tip 26 will be in direct contact with the endocardium or other tissue when in use. When an external magnetic system 54 is activated, the tip 26 will be compressed firmly against the endocardium or other tissue by magnetic force, to anchor the tip 26 in place. Preferably, the tip 26 is magnetically polarized, to thereby generate the magnetic force by which the tip may be anchored in place. For example, the tip 26 is preferably paramagnetic or diamagnetic. If the tip 26 is magnetically polarized, this will create a magnetic field which may react to an external magnetic system 54 thereby anchoring the tip 26 in place.

A catheter 20 in accordance with the present invention may preferably be able to detect electrical signals in the myocardium 52, or other tissue, and to provide electrical signals thereto. Such signals may be detected or provided through an electrode 28 which is positioned in the catheter 20 at the distal end 27 thereof and in contact with the electrically conductive tip 26. The electrode 28 may be implemented, for example, as a platinum disk which is located within the catheter body 22 in contact with the tip 26. However, other materials and shapes may be used to implement the electrode 28. Preferably, the tip 26 extends distally from the inside of the catheter 20, where it is coupled to the electrode 28, through to the outside distal end thereof. The electrode 28 is preferably ferromagnetic and an excellent electric conductor.

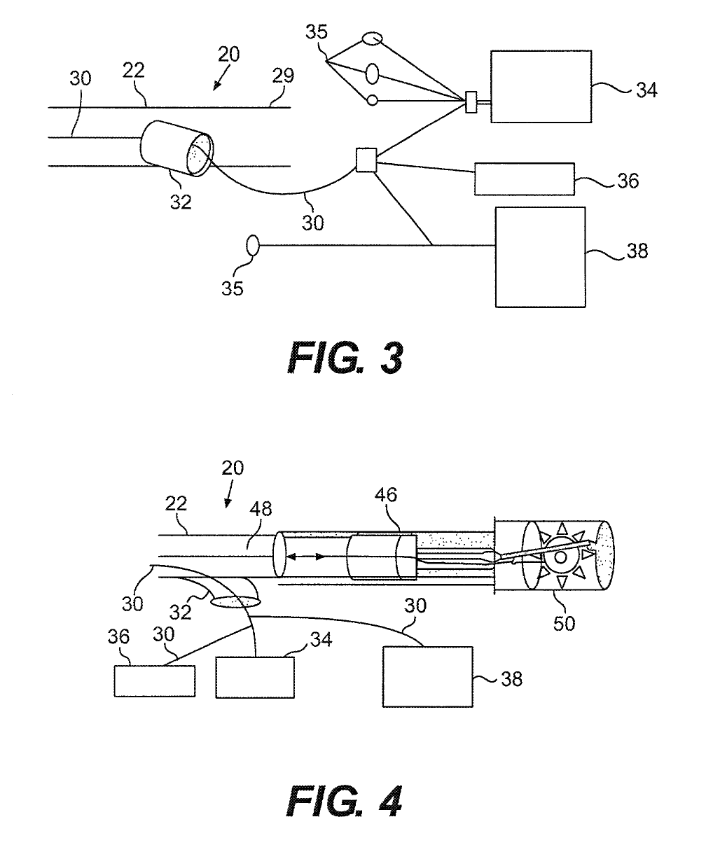

The electrode 28 is coupled to an electrical conductor 30 which is preferably a wire. The wire conductor 30 may be made of copper, or some similar electrically conductive and paramagnetic (or diamagnetic) material. The wire conductor 30 may extend from the electrode 28 through the interior of the catheter 20 to the proximal end 29 thereof. As illustrated, for example, in FIG. 3, the wire conductor 30 connected to the electrode 28 may exit the catheter body 22 at or near a proximal end 29 thereof through a wire port 32 formed in the catheter body 22.

The wire conductor 30 may be connected to one or more electrical devices for detecting electrical signals at the electrode 28 or for providing electrical signals to the electrode 28. Such electrical devices may include, for example, an EKG device 34 for monitoring an endocardial EKG signal detected at the electrode 28, or other cardiac signal monitoring devices, such as a monophasic action potential detector.

For monophasic action potential applications, an additional electrode with a conductive wire would be incorporated into the catheter tip 26 and would be used as a positive electrode. Moreover, the monophasic action potential electrode would be electrically insulated from the electrode 28 (of the catheter 20) and the catheter tip 26. This monophasic action potential electrode could be made of a platinum rod and be designed to be depressed into the endocardium when the tip 26 is firmly engaged against the endocardium. The catheter 20 electrode 28 would then be used as a negative electrode.

As will be discussed in more detail below, an EKG 34 (or other monitoring device), with patches 35 (which are attached to the body of the patient 300 and which function as a ground or reference), may be used to monitor a region of the myocardium 52 against which the tip 26 of the catheter 20 is positioned. The monitoring device may be used to confirm ischemia of such a specified region. Electrical devices for providing pacing 36 and/or defibrillation 38 signals to the myocardium 52 via the electrode 28 may also be connected to the wire conductor 30. Patches 35, which again may function as a ground or reference, can be used in conjunction with the pacing device 36 or the defibrillating device 38.

If during use of the catheter 20 to perform, reperfusion of an ischemic myocardium, a heart block or a ventricular arrhythmia (such as ventricular tachycardia or ventricular fibrillation) develops, pacing and/or defibrillation may be provided by the devices 36 or 38 directly to the myocardium 52 via the electrode 28. As the electrode 28 is in direct electrical contact, via the tip 26, with the myocardium 52, such pacing and/or defibrillation energy may be provided effectively with a minimal required electric output. Note that such arrhythmias, requiring pacer 36 or defibrillator 38 intervention, may be detected by an EKG device 34 connected to the patient 300 externally or by the EKG device 34 monitoring the cardiac activity signals provided by the myocardial electrode 28.

Returning to FIG. 1, the catheter 20 preferably includes one or more exit ports 40 formed near the distal end 27 thereof. The exit ports 40 may preferably be formed slightly proximally of the catheter tip 26 of the catheter 20. For example, the exit ports 40 may be formed approximately two millimeters proximal of the distal end 27 of the catheter tip 26 of the catheter 20. The exit ports 40 are preferably distributed radially around the catheter body 22, and open in a generally distal direction. One or more needles 42 are positioned in the catheter body 22 proximal to the exit ports 40. Preferably at least one needle 42 is provided for each exit port 40. The needles 42 are moveably mounted within the catheter body 22 so as to be aligned with corresponding exit ports 40. The needles 42 are preferably made of a rigid material, such as relatively hard plastic, and may preferably have sharpened tips which are capable of penetrating tissue, such as the endocardium.

The needles 42 are mounted in the catheter body 22 so as to be movable therein in an extending direction, so that the needles 42 may be extended through the exit ports 40 outward from the catheter 20 in a distal direction forward of the catheter tip 26 when the catheter tip 26 is positioned in a desired location. The needles 42 preferably also may be retracted backward into the catheter body 22 from such an extended position. Any method for moving the needles 42 in such a manner, which is operable from the proximal end 29 of the catheter 20, may be employed. Preferably, the needles 42 may be mounted to a mobile disk 44 or other structure mounted within the catheter body 22. By moving the mobile disk 44 forward, the needles 42 will extend outward from the catheter 20 through the exit ports 40. By moving the mobile disk backward (in a proximal direction), the needles 42 will be retracted back into the catheter body 22.

The mobile disk 44 may be moved forward and backward within the catheter body 22 in a conventional manner. For example, a somewhat stiff wire may be attached to the disk 44 and extended through the catheter body 22 to the proximal end thereof. An operator may move the stiff wire backward and forward to thereby move the disk 44, and the needles 42 attached thereto, in the extending and retracting directions.

Alternatively, and preferably, the mobile disk 44 may be moved backward and forward in the catheter 20 hydraulically. In such a case, the diameter of the mobile disk 44 is selected to be an appropriate size such that the outer circumference of the disk 44 forms a moving seal with the inner diameter of the catheter body 22. As illustrated in FIG. 4, a piston 46 is positioned at the proximal end of the catheter body 22. The lumen space 48 within the interior of the catheter body 22, between the mobile disk 44 and the piston 46, is filled with a fluid, such as heparinized normal saline.

The piston 46 is mechanically coupled to an oscillator 50, or operated manually, to move the piston 46 forward and backward. As the piston 46 is moved forward, a positive pressure is generated inside the fluid filled lumen 48 of the catheter 20; this positive pressure forces the mobile disk 44 to advance thereby extending the needles 42 through the exit ports 40 from the catheter body 22. Correspondingly, as the piston 46 is moved backward, a negative pressure is generated within the catheter lumen 48 thereby retracting the mobile disk 44 and the needles 42 attached thereto.

The needles 42 are preferably of sufficient length such that when the needles 42 are moved into the extended position (such as by forward movement of the mobile disk 44), the distal ends of the needles 42 will extend beyond the tip 26 of the catheter 20. For example, for percutaneous trans-endocardial reperfusion applications, and where the exit ports 40 are positioned approximately two millimeters proximal the tip 26, the needles 42 may be, for example, approximately seven millimeters in length. Therefore, when the mobile disk 44 is moved fully forward, such that the needles 42 are fully extended from the exit ports 40, the needles 42 will extend approximately five millimeters beyond the distal tip 26 of the catheter 20. Thus, when the distal tip 26 of the catheter 20 is positioned against the endocardium, or other tissue, the needles 42 will bore into the endocardium, in this example, to a depth of approximately five millimeters.

For percutaneous trans-endocardial reperfusion, the needles 42 are preferably selected to have an outer diameter which is sufficient to form large enough channels in the myocardium 52 to provide reperfusion thereof, and such that the channels remain open for sufficient time to provide such reperfusion until a more permanent treatment can be provided. For example, the needles 42 may have an outer diameter of approximately 0.5 millimeters. It should be understood, however, that the needles 42 may be selected to have any length and/or diameter as may be appropriate for a particular application.

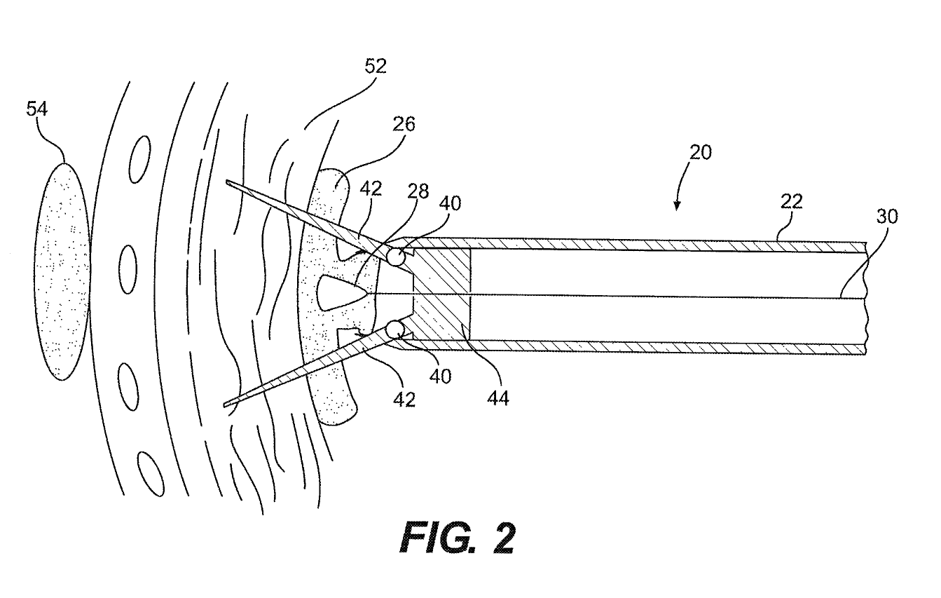

The schematic illustration of FIG. 2 shows the exemplary percutaneous trans-endocardial reperfusion catheter 20 of FIG. 1 with the needles 42 in a fully extended position to form channels to provide reperfusion of an ischemic region of the myocardium 52. As will be discussed in more detail below, in operation to perform percutaneous transendocardial reperfusion, the tip 26 of the catheter 20 is positioned against a portion of the myocardium 52 to be treated. A magnetic system 54, positioned external to the patient, is activated to generate a magnetic field which pulls the ferromagnetic tip 26 of the catheter 20 against the myocardium 52. The force of the magnetic field thus compresses the sponge tip 26 firmly against the myocardium 52 to anchor the tip 26 in place.

The mobile disk 44 is then moved forward by operation of the oscillator 50 and piston 46. Forward movement of the mobile disk 44 causes the needles 42 to move forward and outward from the exit ports 40. The needles 42 are thus extended forward of the distal catheter tip 26 of the catheter 20, piercing through the penetrable catheter tip 26, if necessary, and into the myocardium 52 to form channels therein. The mobile disk 44 may be moved forward and backward several times, by operation of the oscillator 50 and piston 46, to effectively drill multiple channels into the myocardium 52. The channels thereby formed in the myocardium 52 provide the required reperfusion of the myocardium 52.

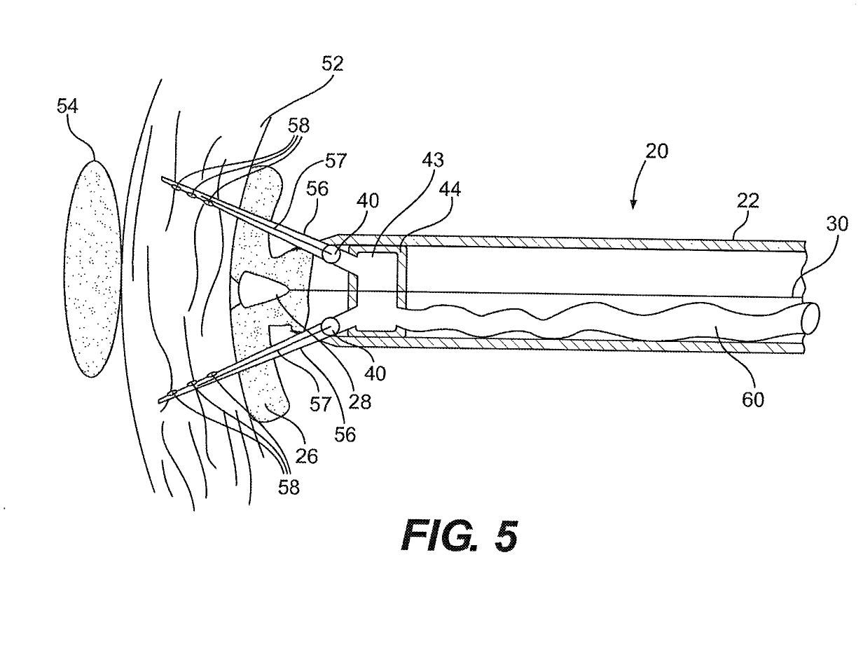

A percutaneous trans-endocardial reperfusion catheter 20 in accordance with the present invention may also be employed to deliver drugs or other fluids directly to the myocardium 52 or other tissue. For example, as illustrated in FIG. 5, one or more hollow needles 56 which are provided in the percutaneous trans-endocardial reperfusion catheter 20 may have a central channel 57 formed therein. The hollow needles 56 are similar to the needles 42 shown in FIG. 1 but are slightly different due to their hollow nature. The channel 57 formed in the hollow needles 56 is in fluid communication with one or more needle holes 58 formed at or near the distal ends of such needles 56.

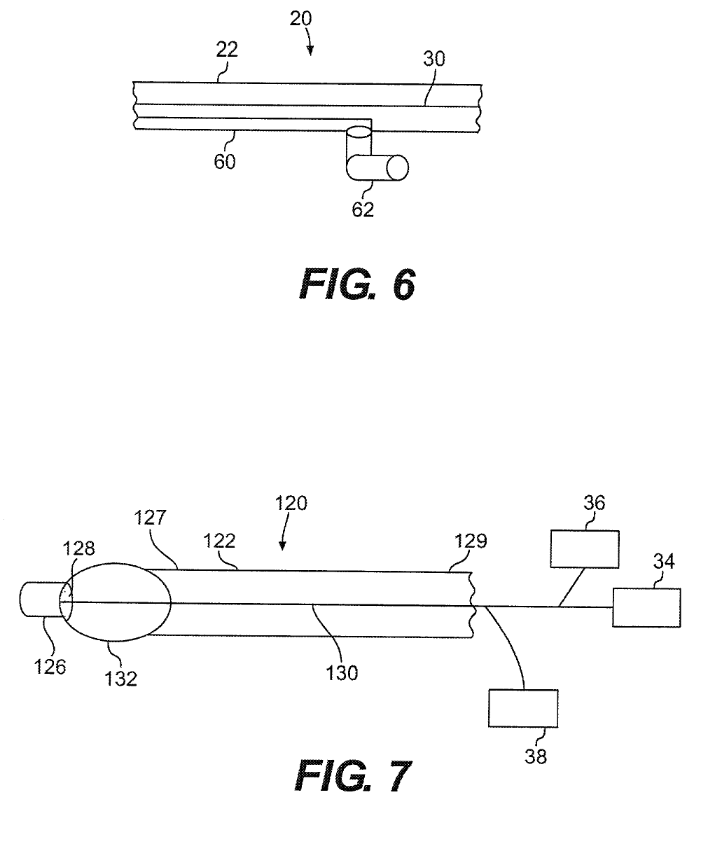

Preferably, several needle holes 58 may be formed in the hollow needles 56 (via a cavity 43 in the disk 44) so as to be in fluid communication with the needle channels 57. The holes 58 may extend through a side of the needles 56 near the distal ends thereof. At the proximal end of the hollow needles 56, i.e., where the needles 56 are attached to the mobile disk 44, a hollow tubule 60 may be connected to be, via the cavity 43 in the disk 44, in fluid communication with the channels 57 formed in the needles 56. As shown in FIG. 6, the tubule 60 preferably extends through the body 22 of the catheter 20 to a tubule port 62 which may extend from a side of the catheter body 22. Drugs, blood, or other fluids may be injected into the tubule 60 through the tubule port 62.

As illustrated in FIG. 5, when the hollow needles 56 are moved into the extended position, such that the needles 56 penetrate into the myocardium 52 (or other tissue), the holes 58 formed in the hollow needles 56 will be positioned within the myocardium 52 (or other tissue). Thus, drugs or other fluids injected into the tubule port 62 will flow through the tubule 60 into the channels 57 formed in the hollow needles 56 and out of the holes 58 formed therein directly into the myocardium 52 or other tissue. Moreover, any regular injection syringe may be used for injecting a drug into the tubule port 62.

Alternatively, an infusion pump (not shown) or other device may be connected to the tubule port 62 to provide a continues flow of drugs, blood, or other fluid directly to the myocardium 52 (or other tissue) through the tubule 60, hollow needle 56, and holes 58. The tubule port 62 may, alternatively, be open to an intra-arterial cannula, or the ascending aorta, to provide a continuous flow of blood directly from the systemic arterial circulation of the patient 300 through the tubule 60, hollow needle 56, and holes 58 to an ischemic portion of the myocardium 52 to provide enhanced reperfusion thereof.

A compressive, ferromagnetic material, as previously described, may be employed, in combination with an external magnetic system 54, to anchor the distal end 27 of a catheter 20 in place against the endocardium or other tissue to be monitored and/or treated. An exemplary alternative embodiment of a catheter 120 employing such a feature is illustrated in, and will be described in detail with reference to, FIG. 7. The exemplary catheter 120 illustrated in FIG. 7 may be employed, for example, for monitoring electrical activity of a chamber in the heart such as the right and left ventricles, and providing electrical therapy thereto, as may be needed.

The catheter 120 includes a catheter body 122, which defines an exterior and an interior of the catheter 120. A compressive, ferromagnetic tip 126 is positioned at the distal end 127 of the catheter 120. As previously discussed, the tip 126 is preferably electrically conductive, may be magnetically polarized, and may be made of a material such as an iron sponge material. An electrode 128, which may be a platinum disk, is coupled to the tip 126. The electrode 128 is connected to a conductor wire 130. The wire 130 extends through the interior of the catheter 120 to a proximal end 129 thereof, where the wire 130 may be coupled to various electronic monitoring and/or treatment devices. For example, as previously discussed, the wire 130 may extend through a wire port (not shown in FIG. 7) from the catheter body 122 of the catheter 120 and be connected to a monitoring device such as an EKG monitoring device 34, or a monophasic action potential device. The wire 130 may also be connected to electrical treatment devices such as a pacer 36 and/or a defibrillator 38.

The catheter 120 may include a portion thereof which is formed as an inflatable balloon 132. The inflatable balloon 132 portion of the catheter 120 may be implemented and inflated and deflated in a conventional manner. The inflatable balloon 132 is preferably positioned immediately proximal to the magnetic tip 126 of the catheter 120. The catheter 120 illustrated in FIG. 7 may be introduced through a jugular or clavicular vein, in the same manner as a Swan-Ganz catheter. Once inserted, the balloon 132 may be inflated, and the catheter 122 may be flow-directed to the desired chamber of the heart such as the right ventricle. Once positioned in the heart, or other portion of the body, the external magnetic system 54 may be activated, as previously described.

When sufficient magnetic force is applied, the tip 126 will be compressed firmly against the endocardium, or other tissue, and will be anchored in place. The condition of the heart may thereby be monitored, by a monitoring device such as an EKG device 34, via signals picked-up by the electrode 128 through the conductive ferromagnetic tip 126. If irregular heart activity, such as ventricular fibrillation, ventricular tachycardia, and/or complete heart block are detected, appropriate pacing or defibrillating electrical energy may be applied from a pacer 36 and/or a defibrillator 38 to the myocardium 52 via the electrode 128. Such cardiac activity irregularities are life threatening complications which can occur during acute coronary events or cardiac interventional procedures. Thus, the exemplary catheter 120 may be particularly useful in such applications.

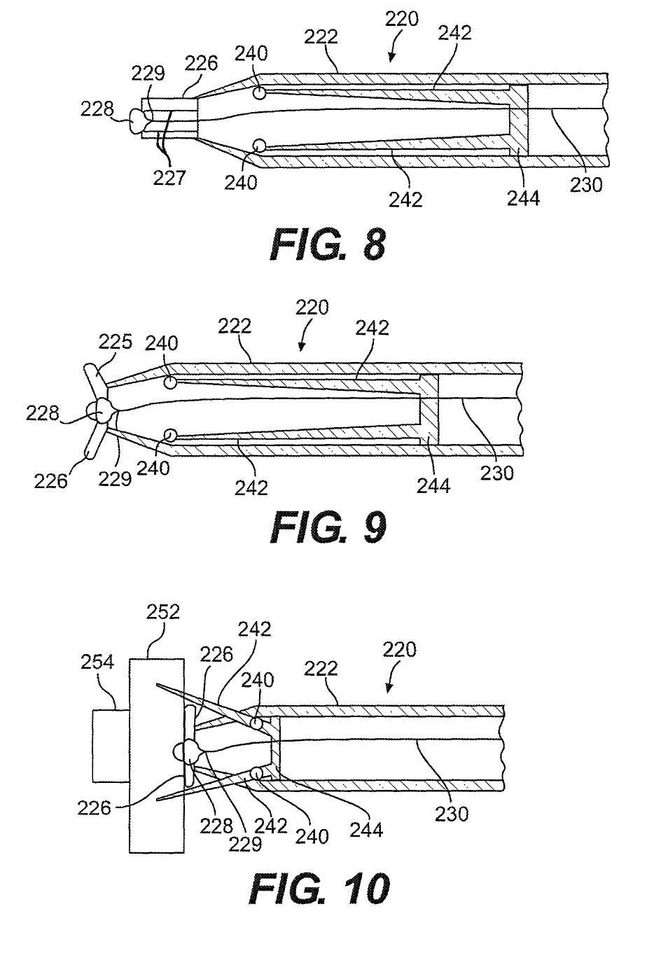

Another alternative embodiment of a percutaneous trans-endocardial reperfusion catheter 220 in accordance with the present invention is illustrated in FIGS. 8-10, and will be described in detail with reference thereto. The alternative embodiment percutaneous trans-endocardial reperfusion catheter 220 is similar to the catheter 20 described previously, with reference to FIG. 1. The alternative embodiment catheter 220 includes a catheter body 222, exit ports 240 formed therein, and one or more needles 242 attached to a structure for extending the needles 242 through the exit ports 240 (to an extended position wherein the needles 242 extend beyond the distal end of the catheter 220); such a needle 242 extending structure may be a mobile disk 244.

This alternative embodiment catheter 220 also includes a ferromagnetic and compressive tip 226 which is attached to the catheter 220 at the distal end thereof. As previously discussed, the compressive, ferromagnetic tip 226 may be formed from an iron sponge material. The compressive, ferromagnetic tip 226 preferably has a plurality of slits 227 formed therein. The slits 227 extend longitudinally and may be distributed radially around the ferromagnetic tip 226 to divide the compressive, ferromagnetic tip 226 into a plurality of sections. Thus, when the compressive, ferromagnetic tip 226 is compressed firmly against the endocardium, or other tissue, by application of an external magnetic system 254, as previously discussed, the ferromagnetic tip material will splay outward, as illustrated in FIG. 9.

In this embodiment, an electrode 228 may be positioned on the exterior of the catheter body 222, in a position distal to and in contact with the compressive tip 226. As previously discussed, the electrode 228 may be formed of a conductive material, such as platinum. The electrode 228 is attached to a conductor wire 230, which extends through the catheter body 222 to a proximal end thereof, where the wire 230 may be connected to various electrical sensing and/or treatment devices. In this case, the proximal side of the electrode 228 is preferably shaped to form a wedge 229, or other similar structure. Thus, when the distal tip 226 of the catheter 220 is compressed firmly against the myocardium 252, or other tissue, the electrode 228 will be pushed backward and the wedge shaped (pointed) side 229 of the electrode 228 will assist in splaying the sections of the compressive tip 226 outward, as illustrated in FIGS. 9 and 10.

The alternative embodiment catheter 220 illustrated in FIGS. 8-10 may be employed, in the manner previously described, for percutaneous trans-endocardial reperfusion, as well as for drug or other fluid delivery and/or cardiac activity or other electrical signal monitoring and treatment. The alternative embodiment electrode 228 and conductor wire 230 may be removable from catheter 220 and may be used as a guide wire for the insertion of the catheter 220.

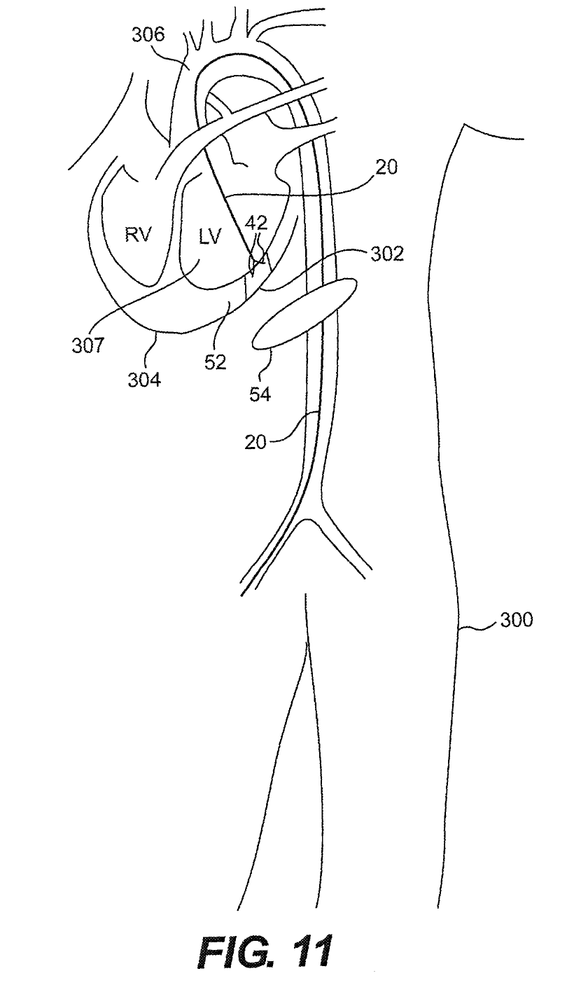

A general procedure for employing a percutaneous trans-endocardial reperfusion catheter 20 in accordance with the present invention for the treatment of acute myocardial ischemia will be described in detail with reference to FIG. 11. However, as previously discussed, it should be understood that a catheter 20 in accordance with the present invention may be employed using other procedures and may be used to perform therapies other than percutaneous trans-endocardial reperfusion for the treatment of acute myocardial ischemia.

The exemplary procedure may be performed on a patient 300 who is preferably positioned on a procedure table which is designed to allow C-arm fluoroscopy. The procedure table should be made of magnetic proof materials, so as not to interfere with operation of the external magnetic system 54. A standard percutaneous technique may be used to introduce the catheter 20 into an artery. A conventional introducer set (size 8 or 9 French) may be used. In the exemplary application shown in FIG. 11, the catheter 20 is not flow directed, therefore, fluoroscope or ECHO guidance must be used to direct the catheter 20 to the desired location.

A standard chest wall twelve lead EKG system may be used to determine the approximate location of an ischemic portion 302 of the myocardium 52 of a patient's heart 304. Based on this initial EKG information, the ischemic endocardium is identified, and the tip of the catheter 20 is directed toward the targeted portion 302 of the myocardium 52. The catheter tip 26 may be guided toward the target area 302 by controlling the highly flexible gooseneck 24 portion of the catheter 20 in a conventional manner.

The external magnetic device 54 may be an electromagnetic device which may be employed for navigating the ferromagnetic tip 26 of the catheter 20 into the desired position within the heart 304. The external magnetic device 54 is preferably incorporated in a mobile device. The device 54 may include plural sets of magnets with adjustable strength. The magnets in the device 54 may be activated intermittently and may be activated in coordination with other magnetic devices 54 positioned on different portions of the body surface to change the direction of the catheter tip to navigate the catheter into the desired position 302. In this manner, the catheter 20 may be directed through the aorta 306, into the left ventricle 307 of the heart 304, into an initial position adjacent to the endocardium. Having positioned the tip of the catheter 20 in an initial position, the external magnetic device 54, placed in a position and direction corresponding to the targeted region 302 of the left ventricle 307 may be activated. Activation of the external magnetic device 54 forces the compressive, ferromagnetic tip 26 of the catheter 20 firmly against the targeted portion 302 of the myocardium 52.

An endocardial EKG, derived from electrical signals picked up by the electrode 28 positioned in the catheter 20, may be continuously displayed on an EKG device 34. Characteristic ischemic ST changes, identified from the continuously displayed EKG signal, may be used to confirm the ischemia of the specific region 302. If the ischemia of the specific region 302 is confirmed, the needles 42 may be extended from the exit ports 40 into the myocardium 52 to form channels therein. This may be accomplished by activating (either manually or mechanically) the oscillator 50 which, as previously discussed, may move the piston 46 back and forth thereby moving, in turn, the mobile disk 44 to which the needles 42 are attached. The movement of the disk 44 will cause the needles 42 to extend and retract from the exit ports 40.

The catheter tip 26 may be repositioned, ischemia of a new region confirmed, and channels formed in the ischemic myocardium 52 by the needles 42 in the manner previously described. These steps may be repeated to create channels covering sufficient endocardial areas affected by the ischemia until ischemic symptoms are relieved.

While the needles 42 are engaged in the myocardium 52, drugs, blood, and/or other fluids may be injected through the tubule injection port 62 by a syringe. The drugs may be delivered through the tubule 60, through the channels 57 formed in hollow needles 56, out the needle holes 58, and directly into the myocardium 52. Continuous perfusion of blood through the hollow needles 56 may be provided, by connecting the tubule port 62 to an infusion pump, intra-arterial cannula, or the ascending aorta.

As has been illustrated and described, the present invention provides a percutaneous trans-endocardial reperfusion catheter which is relatively easy to use. Therefore, the percutaneous trans-endocardial reperfusion catheter of the present invention may be employed in most primary care settings, by non-specialists, for the immediate treatment of acute myocardial ischemia. The catheter features described herein may also be applied in other combinations and applications and to provide other therapies.

Although the aforementioned described various embodiments of the invention, the invention is not so restricted. The foregoing description is for exemplary purposes only and is not intended to be limiting. Accordingly, alternatives which would be obvious to one of ordinary skill in the art upon reading the teachings herein disclosed, are hereby within the scope of this invention. The invention is limited only as defined in the following claims and equivalents thereof.

what is claimed is:

1. A catheter comprising:
a catheter body having a distal end and a proximal end, the catheter body defining an exterior and an interior of the catheter; and
a compressive, ferromagnetic material mounted on the distal end of the catheter body to form a catheter tip.

2. The catheter of claim 1, wherein the compressive, ferromagnetic material is a sponge material.

3. The catheter of claim 2, wherein the catheter tip is magnetically polarized in response to an external magnetic field.

4. The catheter of claim 1, further comprising:
an electrical conductor, wherein the compressive, ferromagnetic material is electrically conductive, wherein the compressive, ferromagnetic, and electrically conductive material projects from the interior to the exterior of the catheter at the distal end of the catheter body to form the catheter tip, wherein the conductor is electrically coupled to a portion of the compressive, ferromagnetic, and electrically conductive material, and wherein the conductor extends into the interior of the catheter body.

5. The catheter of claim 4, wherein the electrical conductor includes a platinum disk electrically coupled to the portion of the compressive, ferromagnetic, and electrically conductive material, wherein the platinum disk is in the interior of the catheter, and wherein the electrical conductor includes a wire which extends proximally from the platinum disk to the proximal end of the catheter.

6. The catheter of claim 5, wherein the platinum disk is magnetically polarized in response to an external magnetic field.

7. The catheter of claim 5, wherein the wire is paramagnetic or diamagnetic, and wherein the wire is electrically conductive.

8. The catheter of claim 1, wherein the compressive, ferromagnetic material includes a plurality of slits formed therein such that the catheter tip is splayed open when compressed.

9. The catheter of claim 8, further comprising:
an electrode mounted on a distal end of the compressive, ferromagnetic material, wherein the electrode has a proximal side formed as a wedge, and wherein the electrode splays open the compressive, ferromagnetic material when the electrode is compressed against the compressive, ferromagnetic material.

10. The catheter of claim 1, further comprising:
an inflatable balloon mounted on the catheter body proximal to the catheter tip.

11. The catheter of claim 10, wherein the inflatable balloon is mounted proximally adjacent to the catheter tip.

12. The catheter of claim 1, further comprising:
at least one exit port formed in the catheter body proximal to the catheter tip; and
at least one needle moveably mounted in the catheter body and extendable from the at least one exit port such that the distal end of the at least one needle is adapted to move from a position in the interior of the catheter to a position distally forward of the distal end of the catheter body.

13. The catheter of claim 12, wherein the catheter tip is penetrable by the at least one needle, and wherein the at least one needle is extendable from the at least one exit port such that the distal end of the at least one needle extends distally forward of the catheter tip and projects through the catheter tip.

14. The catheter of claim 12, wherein the at least one exit port is a plurality of exit ports formed in the catheter body proximal to the catheter tip, and wherein the at least one needle is a plurality of needles corresponding to each of the exit ports.

15. A catheter comprising:
a catheter body having a distal end and a proximal end, the catheter body defining an interior and an exterior of the catheter;
a fixation structure positioned at or near the distal end of the catheter;
at least one exit port formed in the catheter body at or near the distal end thereof; and
at least one needle moveably mounted in the catheter body and extendable from the at least one exit port such that the distal end of the at least one needle is adapted to move from a position in the interior of the catheter to a position distally forward of the distal end of the catheter body.

16. The catheter of claim 15, wherein the fixation structure includes a ferromagnetic material mounted on the distal end of the catheter body to form a catheter tip.

17. The catheter of claim 16, wherein the ferromagnetic material is compressive.

18. The catheter of claim 17, wherein the compressive, ferromagnetic material is an iron sponge material.

19. The catheter of claim 17, wherein the catheter tip is penetrable by the at least one needle, and wherein when the at least one needle is extended from the at least one exit port distally forward of the catheter tip, the at least one needle projects through the catheter tip.

20. The catheter of claim 16, further comprising:
an electrical conductor, wherein the ferromagnetic material is electrically conductive, wherein the ferromagnetic and electrically conductive material projects from the interior to the exterior of the catheter at the distal end of the catheter body to form the catheter tip, wherein the conductor is electrically coupled to a portion of the ferromagnetic and electrically conductive material, and wherein the conductor extends into the interior of the catheter body.

21. The catheter of claim 16, wherein the ferromagnetic material includes a plurality of slits formed therein such that the ferromagnetic material is splayed open when compressed.

22. The catheter of claim 21, further comprising:
an electrode mounted on a distal end of the ferromagnetic material, wherein the electrode has a proximal side formed as a wedge, and wherein the electrode splays open the ferromagnetic material when the electrode is compressed against the ferromagnetic material.

23. The catheter of claim 15, further comprising:
a tubule, wherein the at least one needle includes a channel and at least one hole, wherein the channel extends from a proximal end of the at least one needle to the at least one hole, wherein the at least one hole is in fluid communication with the channel, wherein a distal end of the tubule is connected to the proximal end of the at least needle, and wherein the tubule is in fluid communication with the channel.

24. The catheter of claim 15, further comprising:
a reciprocator operable from the proximal end of the catheter for extending the at least one needle through the at least one exit port and for retracting the at least one needle into the interior of the catheter.

25. The catheter of claim 15, further comprising:
a disk mounted for reciprocal motion in the interior of the catheter near the distal end of the catheter body;
a piston mounted for reciprocal motion near the proximal end of the catheter body; and
a reciprocater, wherein the at least one needle is attached to a distal side of the disk, wherein fluid fills the interior of the catheter between the disk and the piston, and wherein the reciprocater is adapted to move the piston in the catheter body to create pressure within the catheter body which moves the disk forward and backward thereby extending the at least one needle through the at least one exit port and retracting the at least one needle into the interior of the catheter.

26. The catheter of claim 25, wherein the fluid which fills the interior of the catheter is heparinized normal saline.

27. The catheter of claim 25, wherein the reciprocater is an oscillator.

28. The catheter of claim 15, wherein the at least one exit port is a plurality of exit ports formed in the catheter body at or near the distal end of the catheter body, and wherein the at least one needle is a plurality of needles corresponding to each of the exit ports.

29. A catheter comprising:
a catheter body having a distal end and a proximal end, the catheter body defining an interior and an exterior of the catheter;
at least one exit port formed in the catheter body at or near the distal end of the catheter body;
at least one needle moveably mounted in the catheter body and extendable from the at least one exit port such that a distal end of the at least one needle extends forward of the distal end of the catheter body, wherein the at least one needle includes a channel extending from a proximal end of the at least one needle to at least one hole formed in the at least one needle, the hole being in fluid communication with the channel; and
a tubule having a distal end connected to a proximal end of the channel, the tubule being in fluid communication with the channel.

30. The catheter of claim 29, further comprising:
a fixation structure positioned at or near the distal end of the catheter body.

31. The catheter of claim 30, wherein the fixation structure includes a ferromagnetic material mounted on the distal end of the catheter body to form a catheter tip.

32. The catheter of claim 31, wherein the catheter tip is penetrable by the at least one needle, and wherein the at least one needle is extendable from the at least one exit port such that the distal end of the at least one needle extends forward of the catheter tip and projects through the catheter tip.

33. The catheter of claim 31, further comprising:
an electrical conductor, wherein the ferromagnetic material is electrically conductive, wherein the ferromagnetic and electrically conductive material projects from the interior to the exterior of the catheter at the distal end of the catheter body to form the catheter tip, wherein the conductor is electrically coupled to a portion of the ferromagnetic and electrically conductive material, and wherein the conductor extends into the interior of the catheter body.

34. The catheter of claim 31, wherein the ferromagnetic material includes a plurality of slits formed therein such that the ferromagnetic material is splayed open when compressed.

35. The catheter of claim 34, further comprising:
an electrode mounted on a distal end of the ferromagnetic material, wherein the electrode has a proximal side formed as a wedge, and wherein the electrode splays open the ferromagnetic material when the electrode is compressed against the ferromagnetic material.

36. The catheter of claim 29, wherein the at least one needle includes at least one hole in fluid communication with the channel, and wherein the at least one hole is formed in a side of the at least one needle.

37. The catheter of claim 36, wherein the at least one needle includes a plurality of holes in fluid communication with the channel, and wherein the holes are formed in a side of the at least one needle.

38. The catheter of claim 29, wherein the tubule extends from the proximal end of the at least one needle through the interior of the catheter.

39. The catheter of claim 29, further comprising:
a tubule port formed in the catheter body which is in fluid communication with the tubule.

40. The catheter of claim 29, further comprising:
a reciprocator operable from the proximal end of the catheter for extending the at least one needle through the at least one exit port and for retracting the at least one needle into the interior of the catheter.

41. The catheter of claim 29, further comprising:
a disk mounted for reciprocal motion in the interior of the catheter near the distal end of the catheter body;
a piston mounted for reciprocal motion near the proximal end of the catheter body; and
a reciprocator, wherein the at least one needle is attached to a distal side of the disk, wherein fluid fills the interior of the catheter between the disk and the piston, and wherein the reciprocator is adapted to move the piston in the catheter body to create pressure within the catheter body which moves the disk forward and backward thereby extending the at least one needle through the at least one exit port and retracting the at least one needle into the interior of the catheter.

42. The catheter of claim 41, wherein the fluid which fills the inside of the catheter body is heparinized normal saline.

43. The catheter of claim 41, wherein the reciprocater is an oscillator.

44. The catheter of claim 29, wherein the at least one exit port is a plurality of exit ports formed in the catheter body at or near the distal end thereof, and wherein the at least one needle is a plurality of needles corresponding to each of the exit ports.

45. A catheter comprising:
a catheter body having a distal end and a proximal end, the catheter body defining an interior and an exterior of the catheter;
at least one exit port formed in the catheter body at or near the distal end of the catheter body;
at least one needle mounted in the catheter body and extendable from the at least one exit port such that a distal end of the at least one needle extends distally forward of the distal end of the catheter body;
a disk mounted for reciprocal motion in the interior of the catheter near the distal end of the catheter body, wherein the at least one needle is attached to a distal side of the disk;
a piston mounted for reciprocal motion near the proximal end of the catheter body; and
fluid which fills the interior of the catheter between the disk and the piston.

46. The catheter of claim 45, further comprising:
a fixation structure positioned at or near the distal end of the catheter.

47. The catheter of claim 46, wherein the fixation structure includes a ferromagnetic material mounted on the distal end of the catheter body to form a catheter tip.

48. The catheter of claim 47, wherein the catheter tip is penetrable by the at least one needle, and wherein the at least one needle is extendable from the at least one exit port such that the distal end of the at least projects through the catheter tip.

49. The catheter of claim 47, further comprising:
an electrical conductor, wherein the ferromagnetic material is electrically conductive, wherein the ferromagnetic and electrically conductive material projects from the interior to the exterior of the catheter at the distal end of the catheter body to form the catheter tip, wherein the conductor is electrically coupled to a portion of the ferromagnetic and electrically conductive material, and wherein the conductor extends into the interior of the catheter body.

50. The catheter of claim 47, wherein the ferromagnetic material includes a plurality of slits formed therein such that the ferromagnetic material is splayed open when compressed.

51. The catheter of claim 50, further comprising:
an electrode mounted on a distal end of the ferromagnetic material, wherein the electrode has a proximal side formed as a wedge, and wherein the electrode splays open the ferromagnetic material when the electrode is compressed against the ferromagnetic material.

52. The catheter of claim 45, further comprising:
a tubule, wherein the at least one needle includes a channel and at least one hole, wherein the channel extends from a proximal end of the at least one needle to the at least one hole, wherein the at least one hole is in fluid communication with the channel, wherein a distal end of the tubule is connected to the proximal end of the at least needle, and wherein the tubule is in fluid communication with the channel.

53. The catheter of claim 45, wherein the fluid which fills the interior of the catheter is heparinized normal saline.

54. The catheter of claim 45, further comprising:
a reciprocater for moving the piston in the catheter body to create pressure within the catheter body which moves the disk forward and backward and thereby extends the at least one needle from the at least one exit port and retracts the at least one needle into the interior of the catheter.

55. The catheter of claim 54, wherein the reciprocater is an oscillator.

56. The catheter of claim 45, wherein the at least exit port is a plurality of exit ports formed in the catheter body at or near the distal end thereof, and wherein the at least one needle is a plurality of needles corresponding to each of the exit ports.

57. A method for the treatment of acute myocardial ischemia comprising the steps of:
providing a catheter having a distal end in which a plurality of needles are mounted;
positioning the distal end of the catheter within a chamber of a patient's heart adjacent to an ischemic region of the chamber's endocardium;
extending the plurality of needles beyond the distal end of the catheter through the chamber's endocardium to create simultaneously a plurality of holes in the ischemic region of the chamber's myocardium.

58. The method of claim 57, wherein the chamber is the patient's left ventricle, and wherein the step of positioning the distal end of the catheter within a chamber of the heart includes the steps of inserting the distal end of the catheter percutaneously into an artery and directing the distal end of the catheter through the patient's aorta and into the left ventricle.

59. The method of claim 57, further comprising the step of:
confirming an ischemic region of the chamber's endocardium using a signal provided by an electrode before extending the plurality of needles,
wherein the electrode is positioned at the distal end of the catheter.

60. The method of claim 57, further comprising the step of:
anchoring the distal end of the catheter to the ischemic region of the chamber's endocardium before extending the plurality of needles into the chamber's myocardium.

61. The method of claim 60, wherein the catheter includes a ferromagnetic material positioned on the distal end thereof to form a catheter tip, and wherein the step of anchoring the distal end of the catheter includes the step of applying an external magnetic field to force the catheter tip firmly against the endocardium.

62. The method of claim 57, further comprising the step of:
delivering a fluid through a hole in at least one of the plurality of needles and into the myocardium.

63. The method of claim 62, wherein the fluid is a drug or blood.

64. The method of claim 62, wherein the fluid is blood, and wherein the step of delivering fluid through the needle hole and into the myocardium includes the step of establishing fluid communication between the needle hole and an intra-arterial cannula via a tubule.

65. The method of claim 62, wherein the fluid is blood, and wherein the step of delivering fluid through the needle hole and into the myocardium includes the step of establishing fluid communication between the needle hole and the patient's ascending aorta via a tubule.

66. The method of claim 57, further comprising the step of:
applying electrical energy to the heart via an electrode positioned at the distal end of the catheter.

67. The method of claim 66, wherein the step of applying electrical energy to the heart includes the step of applying defibrillating electrical energy to the heart via the electrode.

68. The method of claim 66, wherein the step of applying electrical energy to the heart includes the step of applying pacing electrical energy to the heart via the electrode.
